(19) United States
(12) Patent Application Publication
(10) Pub. No.: US 2006/0195090 A1
(43) Pub. Date: Aug. 31, 2006

(54) Apparatus for and method of aligning a spine

(76) Inventor: Loubert Suddaby, Orchard Park, NY (US)

Correspondence Address: SHOEMAKER AND MATTARE, LTD, 10 POST OFFICE ROAD - SUITE 110, SILVER SPRING, MD, 20910, US

(21) Appl. No.: 11/054,305

(22) Filed: Feb. 10, 2005

(51) Int. Cl. A61B 17/58; A61B 17/56

(52) U.S. Cl. 606/61

(57) ABSTRACT

A plurality of pins attach to bone, such as a vertebra (not shown). Spacers interpose between pins. A cable extends through pins and spacers between a cap and an expansion sleeve. The expansion sleeve increases cable tension to restrict movement among pins and spacers.

### BACKGROUND OF THE INVENTION

This invention relates to the field of orthopedic surgery and particularly to the restoration and preservation of spinal alignment.

Spinal osteosynthesis devices typically include rigid rods that are connected to the vertebrae of the spine with a bone anchor. The rods are bent to accommodate or provide curvature appropriate to the anatomy of a spinal segment. The bone anchors often include screws or pedicle hooks. Sometimes adjacent rods are interconnected transversely.

Accommodating the spine and providing appropriate curvature requires bending the rods in two planes, the sagittal and frontal planes, which is complex and difficult to undertake properly outside of the patient. Bending the rods on the patient during surgery also is difficult and not without risk to the patient.

Spinal osteosynthesis devices typically have a large number of parts. The number and complexity of parts of these devices increases the costs and failure modes thereof. This complexity increases surgical implantation time and cost.

What are needed, and not taught or suggested in the art, are an apparatus for aligning a spine that is easy to implant and configured with few parts, and a method of aligning a spine that promotes reduced device and implantation cost and surgical duration, cost and risk.

### SUMMARY OF THE INVENTION

The invention overcomes the disadvantages noted above and provides an apparatus for aligning a spine that is easy to implant and configured with few parts, and a method of aligning a spine that promotes reduced device and implantation cost and surgical duration, cost and risk.

The invention utilizes a segmental rod construct which can assume a rigid, linear or curvilinear attitude when a central cable is tautened within a series of hollow segments. The hollow segments mate with each other in such a fashion that a solid construct is formed when the component segments are drawn together as the central cable is appropriately tautened. The component segments are constructed to appose and interlock when drawn together by a central cable, which passes through the hollow interior of each component segment.

The interdigitation between component segments is such that they will resist all manner of forces, including compression, distraction, bending and torsion, when the central cable is appropriately tightened. Once the central cable is appropriately tautened and the segments are completely interdigitated and mated, the resulting construct functions in the manner as a solid rigid or semi-rigid rod.

By utilizing segments which are curved to appropriate degrees, spinal curvatures, such as kyphosis, lordosis or scoliosis, can be altered, eradicated or preserved.

An embodiment of an apparatus configured according to principles of the invention includes a plurality of pins, each configured to attach to a bone, such as a vertebra. A plurality of spacers are configured to be interposed between at least two of the pins. A singular means for restricting movement restricts relative movement of all of pins and spacers.

An embodiment of a method configured according to principles of the invention includes attaching to each of a predetermined number of vertebrae one of a like number of pins, interposing a spacer between at least two of the pins, and actuating a means for restricting relative movement among the pins and spacers.

The invention provides improved elements and arrangements thereof, for the purposes described, which are inexpensive, dependable and effective in accomplishing intended purposes of the invention.

Other features and advantages of the invention will become apparent from the following description of the preferred embodiments, which refers to the accompanying drawings.

### BRIEF DESCRIPTION OF THE DRAWINGS

The invention is described in detail below with reference to the following figures, throughout which similar reference characters denote corresponding features consistently, wherein:

FIG. 1 is a left side elevational view, partially in cross-section, of an embodiment of an apparatus for aligning a spine configured according to principles of the invention, having a curved formation;

FIG. 2 is a left side elevational view, partially in cross-section, of the embodiment of FIG. 1, having a straight formation;

FIG. 3 is a front elevational view of the embodiment of FIG. 1;

FIG. 4 is a bottom elevational view of the embodiment of FIG. 1;

FIG. 5 is an enlarged left side elevational view of a pin and spacer of the embodiment of FIG. 1;

FIG. 6 is a perspective view of the embodiment of FIG. 5;

FIG. 7 is a schematic view of an embodiment of a method of aligning a spine configured according to principles of the invention; and

FIGS. 8-13 are left side environmental perspective views of steps of the method of FIG. 7.

### DETAILED DESCRIPTION OF THE PREFERRED EMBODIMENTS

The invention is an apparatus for aligning a spine that is easy to implant and configured with few parts, and a method of aligning a spine that promotes reduced device and implantation cost and surgical duration, cost and risk.

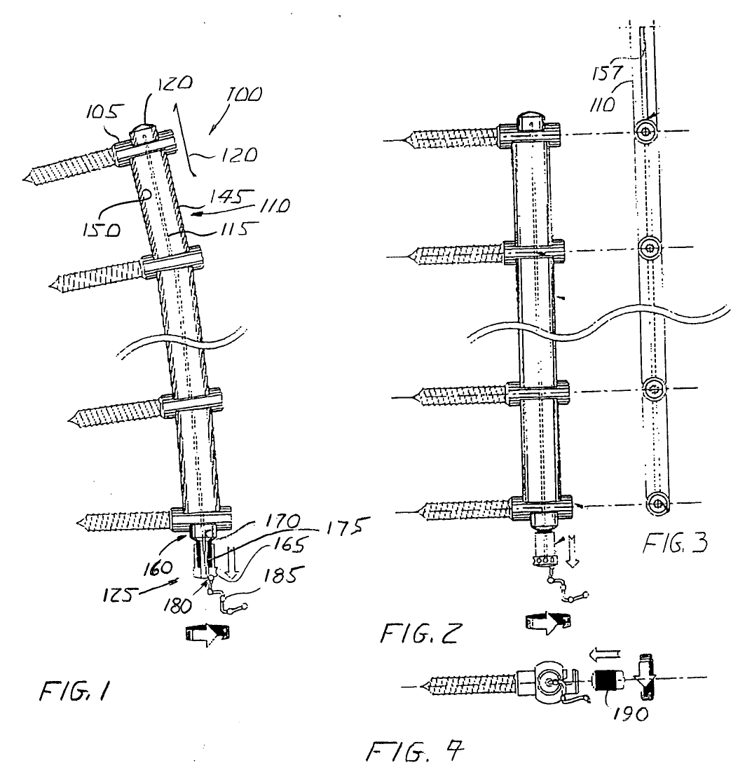

Referring to FIG. 1, an embodiment of an apparatus 100 configured according to principles of the invention includes a plurality of pins 105 configured to attach to bone, such as a vertebra (not shown). Spacers 110 interpose between pins 105. A cable 115 extends through pins 105 and spacers 110 between a cap 120 and an expansion sleeve 125.

Referring also to FIGS. 5 and 6, pin 105 has a head 130, a stem 135 and a passage 140 therethrough. Head 130 has a surface that is knurled or otherwise treated for selective frictional engagement with spacer 110, as described below. Head 130 also has a driver socket (not shown) or other means for being driven into or attached to bone, as described below.

Stem 135 is configured to attach to bone. To this end, stem 135 may have threads, as is common to many bone anchor apparatuses. Stem 135 may assume any configuration suited for the purposes described.

Passage 140 freely receives and passes cable 115. Passage 140 may be coated or otherwise treated to avoid snags or binding of cable 115, which would cause undesired, localized variances in the tension of cable 115, described in greater detail below.

Referring to FIGS. 1-5, spacer 110 has a cylindrical body 145 with a passage 150 and terminal surfaces 155, as best seen in FIG. 5. Body 145 has a length that may be unique relative to the lengths of other bodies 145 defining apparatus 100 as implanted. Passage 150 is aligned with and receives cable 115 from passage 140. Spacer 110 also has a lengthwise slot 157 configured to receive cable 115. Preferably, spacer 110 is constructed of material or configured to allow sufficient deformation to open slot 157 in an amount necessary to receive cable 115, then close slot (not shown) to retain cable 115 and, perhaps, deter introduction of fluids in passage 150. Like passage 140, passage 150 may be coated or otherwise treated to avoid localized snags or binding of cable 115.

Like surface of head 130, surface 155 is knurled or otherwise treated for selective frictional inter-engagement with head 130. When compression between head 130 and surface 155 is low, pin 105 and spacer 110 may move relatively, and when compression between head 130 and surface 155 is high, pin 105 and spacer 110 resist movement.

When in contact, but not sufficiently compressed together, normal forces among pins 105 and spacers 110 are weak and allow for relative movement or adjustment. Increasing compression among pins 105 and spacers 110 increases compression in a normal direction relative to the surfaces of heads 130 and associated contacting surfaces 155, thereby discouraging relative movement among pins 105 and spacers 110.

Cable 115 is configured to provide sufficient slack so that pins 105 and spacers 110, specifically surface of head 130 and surface 140 of spacer 110, move freely. Cable 115 is constructed of material and configured to withstand sufficient tension to cause sufficient compression among heads 130 of pins 105 and associated surfaces 155 of spacers 110. Cable 115 may have nodules 114 at discrete intervals to provide localized enlarged areas, or other suitable convention that promote enhanced frictional engagement.

As mentioned above, passages 140 and 150 are coated or treated, and are aligned and configured in a coordinated fashion to avoid binding cable 115. Binding would cause elevated tension in a portion of cable 115 and, more importantly, slack in another portion of the cable. Undesired cable slack would decrease compression, thus allow movement among associated pins 105 and spacers 110.

Cap 120 is similar to spacer 110 except that cap 120 provides a terminus to which cable 115 firmly secures. Thus, in practice, cap 120 is disposed after an endmost spacer 110, wherefrom cable 115 threads through all of pins 105 and spacers 110 employed for therapy.

Expansion sleeve 125 includes a body 160 and an adjuster 165. Like cap 120, expansion sleeve 125 also provides a terminus to which cable 115 firmly secures. However, unlike with cap 120, cable 115 may be disconnected from sleeve 125, repositioned, then re-connected to sleeve 125 to effect greater or lesser tension as needed.

Body 160 is similar to spacer 110, in that body 160 defines a surface (not shown) similar to surface 140 and a passage 170 similar to passage 150. Body 160 differs from spacer 110 in that body 160 operably connects with adjuster 165 to effect tension in cable 115. For example, body 160 may have a hollow expansion 175 that passes cable 115 to and threadingly engages with adjuster 165. Rotating adjuster 165 relative to body 160 changes relative positioning of body 160 and adjuster 165 along the length of cable 115.

Adjuster 165 has an opening 180 that is configured for selectively seizing cable 115. For example, opening 180 may be wedge shaped, with a wide portion sized to allow passage of a nodule 185 and a narrow portion sized to prevent passage of nodule 185. This convention provides for rough-tuning of tension in cable 115. That is, drawing nodules 185 through the wide portion of opening 180, then nesting a selected nodule 185 in the narrow portion of opening 180 would adjust tension in cable 115 proportional to the spacing between nodules 185.

Adjuster 165 also provides for fine-tuning tension in cable 115. Once cable 115 is fixed relative to adjuster 165, as mentioned above, adjuster 165 is rotated or otherwise actuated to alter relative positioning with respect to body 160 and increase or decrease tension in cable 115. For example, rotating adjuster 165 counterclockwise relative to body 160 would cause adjuster 165 to translate along expansion 175 away from body 160, which would draw more cable 115 into sleeve 125 and/or increase tension thereof in an amount corresponding to the thread pitch of the threads of expansion 175 and rotation imparted to adjuster 165.

Referring to FIG. 4, apparatus 100 includes a driver 190 for driving pin 105 into bone. Driver 190 is configured to engage with the driver socket (not shown) of head 130. Driver 190 also positions pin 105 to assume an appropriate rotational orientation relative to a vertebra as needed for desired therapy.

Alternatively, driver 190 may be configured to be received in passage 140 to rotate and threadingly install pin 105.

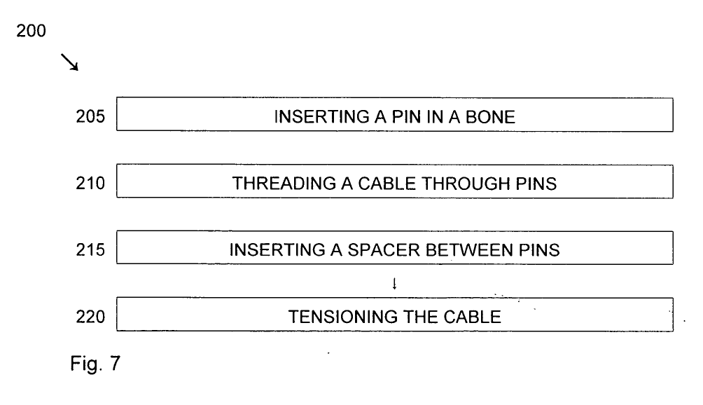

Referring to FIGS. 7-13, an embodiment of a method 200 configured according to principles of the invention includes: a step 205 of inserting a pin in a bone; a step 210 of threading a cable through pins; a step 215 of inserting a spacer between pins; and a step 220 of tensioning the cable. Preferably, method 200 includes: a step 225 of shearing excess cable; and a step 230 of engaging final tension.

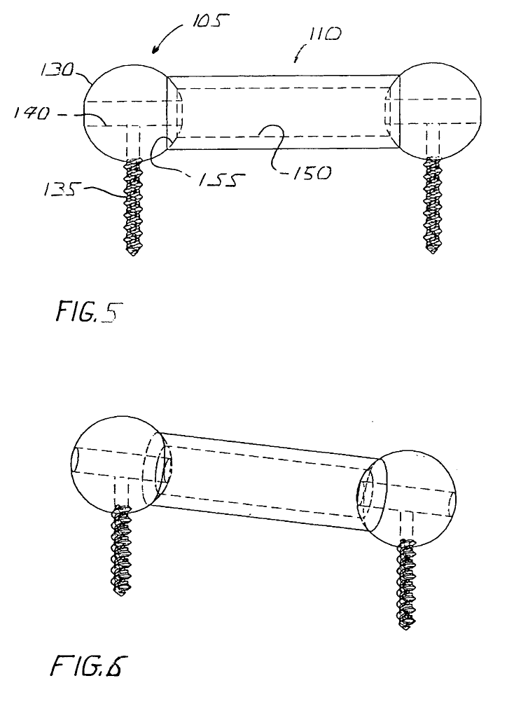

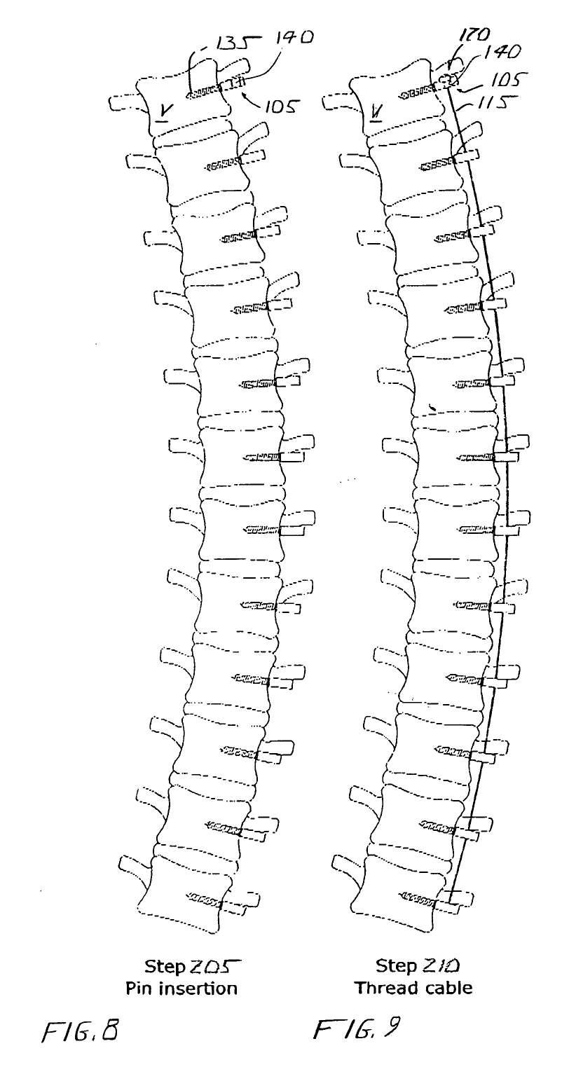

Referring specifically to FIGS. 7 and 8, step 205 of inserting a pin in a bone, preferably, includes a pin 105, described above. A surgeon employs a driver 190, as shown in FIG. 3, that engages pin 105 and threadingly implants stem 135 in a vertebra V. The surgeon repeats step 205 for each vertebra intended for therapy. Each passage 140 of pins 105 implanted are aligned or oriented in a generally linear fashion.

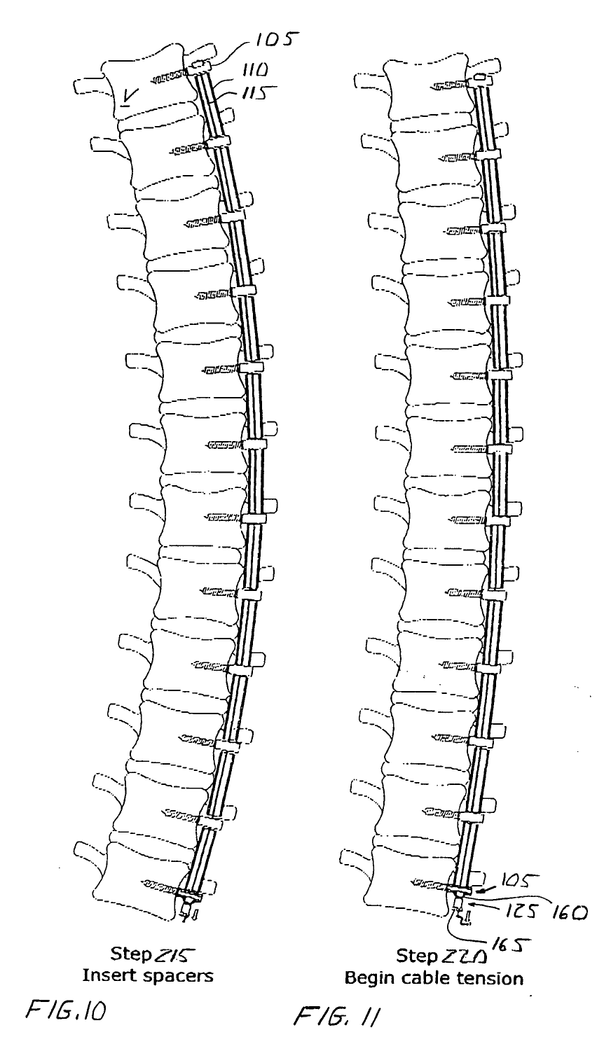

Referring to FIGS. 7 and 9, step 210 of threading a cable through pins, preferably, includes a cable 115. Cable 115 may be fixed to cap 120 and threaded through all of passages 140 of consecutive pins 105 implanted in selected vertebrae V.

Referring to FIGS. 7 and 10, step 215 of inserting a spacer between pins, preferably, includes a spacer 110 interposed between consecutive pins 105. Spacer 110 has a slot 157, as shown in FIG. 3, configured to receive cable 115, as described above. Spacer 110 is deformed enough to open slot 157 sufficiently to permit spacer 110 to slip over cable 115. Opposite surfaces 155 of spacer 110 mate with complementary surfaces of each head 130 of consecutive pins 105, as shown in FIG. 4.

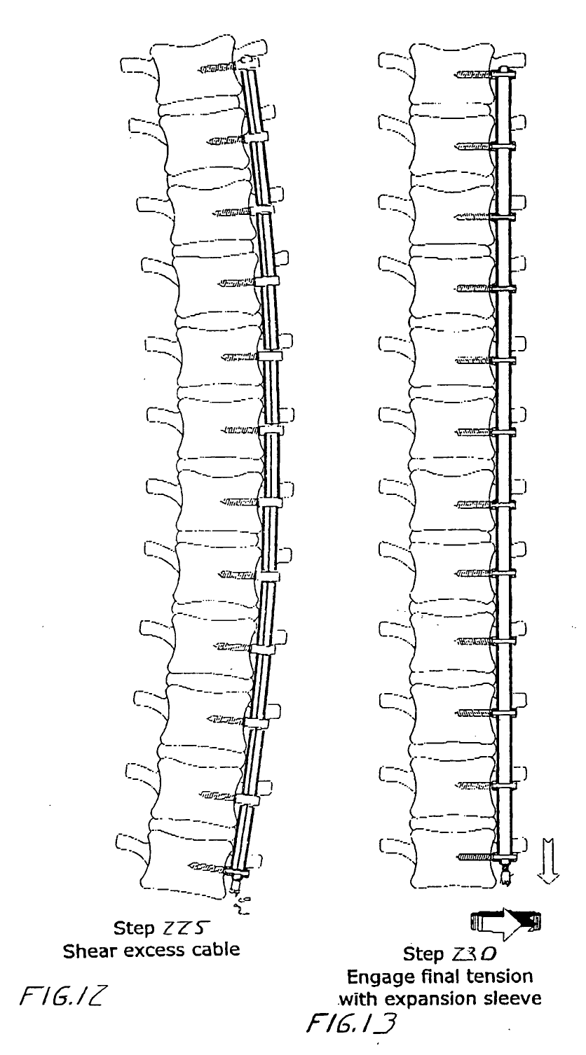

Referring to FIGS. 7 and 11, step 220 of tensioning cable, preferably, includes disposing an expansion sleeve 125 after the last pin 105 and drawing cable 115 through opening 180 thereof. The surgeon pulls nodules 185, disposed only in an end portion of cable 115, through a wide portion of opening 180, then nests a predetermined nodule 185 in a narrow portion of opening 180, providing rough tension in cable 115.

Referring to FIGS. 7 and 12, step 225 of shearing excess cable, while not required for therapy, would be necessary for patient comfort. The surgeon may shear cable 115 in any manner.

Referring to FIGS. 7 and 13, step 230 of engaging final tension, preferably, includes manipulating adjustor 165 and causing adjuster 165 to translate along expansion 175, as shown in FIG. 1, away from body 160. Because the predetermined nodule 185 is nested in a narrow portion of opening 180 in step 220, translating adjuster 165 draws more cable 115 into sleeve 125 and/or increases tension thereof.

The invention is not limited to the particular embodiments described herein, rather only to the following claims.

I claim:

1. Apparatus for aligning a spine comprising:
a plurality of pins, each configured to attach to bone;
a plurality of spacers, each configured to be interposed between at least two of said plurality of pins; and
means for restricting relative movement among said plurality of pins and said plurality of spacers.

2. Apparatus of claim 1, wherein increasing compression among said plurality of pins and said plurality of spacers discourages relative movement thereof.

3. Apparatus of claim 1, wherein said means for restricting relative movement comprises a cable.

4. Apparatus of claim 3, wherein increasing tension in said cable increases compression among said plurality of pins and said plurality of spacers and discourages relative movement thereof.

5. Apparatus of claim 3, wherein each of said plurality of pins and said plurality of spacers define a passage for receiving said cable.

6. Apparatus of claim 3, further comprising an expansion sleeve that is engageable with said cable.

7. Apparatus of claim 6, wherein said expansion sleeve comprises:
means for rough tensioning said cable; and
means for fine tensioning said cable.

8. Apparatus of claim 6, wherein said expansion sleeve comprises an aperture having a wide portion, through which said cable may pass freely, and a narrow portion that selectably frictionally engages said cable.

9. Apparatus for aligning a spine comprising:
a pin configured to attach to bone; and
a spacer configured to contact said pin;
wherein increasing compression between said pin and said spacer discourages relative movement thereof.

10. Apparatus of claim 9, further comprising a cable for effecting compression between said pin and said spacer.

11. Apparatus of claim 10, wherein said pin and said spacer each define a passage for receiving said cable.

12. Apparatus of claim 10, further comprising an expansion sleeve that is engageable with said cable.

13. Apparatus of claim 12, wherein said expansion sleeve comprises:
means for rough tensioning said cable; and
means for fine tensioning said cable.

14. Apparatus of claim 12, wherein said expansion sleeve comprises an aperture having a wide portion, through which said cable may pass freely, and a narrow portion that selectably frictionally engages said cable.

15. Method of aligning a spine comprising:
attaching to each of a predetermined number of vertebrae one of a like number of pins;
interposing a spacer between at least two of the pins; and
actuating a means for restricting relative movement among the pins and spacers.

16. Method of claim 15, wherein increasing compression among the pins and spacers discourages relative movement thereof.

17. Method of claim 15, wherein the means for restricting comprises a cable.

18. Method of claim 17, wherein said actuating a means for restricting comprises manipulating an expansion sleeve that engages the cable and effects tension thereof.

19. Method of aligning a spine comprising:
attaching to each of a predetermined number of vertebrae one of a like number of pins;
interposing a spacer between at least two of the pins; and
increasing compression between the pins and spacers, thereby discouraging relative movement thereof.

20. Method of claim 19, wherein said increasing compression comprises tensioning a cable.

21. Method of claim 20, wherein said tensioning comprises manipulating an expansion sleeve that selectably engages the cable.
